(19) United States
(12) Patent Application Publication
(10) Pub. No.: US 2026/0335313 A1
(43) Pub. Date: Oct. 29, 2026

(54) NOVEL CDK9 INHIBITORS

(71) Applicant: Origenis GmbH, Planegg (DE)

(72) Inventors: Michael Thormann, Planegg (DE); Nasser Yehia, Munchen (DE); Roland Kodtler, Planegg (DE)

(21) Appl. No.: 19/116,737

(22) PCT Filed: Sep. 27, 2023
(86) PCT No.: PCT/EP2023/076719

§ 371 Date: Mar. 28, 2025

(30) Foreign Application Priority Data
Sep. 28, 2022 (EP) 22198460.2

(51) Int. Cl. A61K 31/5025; C07D 487/04

(52) U.S. Cl. CPC A61K 31/5025; C07D 487/04

(57) ABSTRACT

The present invention relates to novel compounds of formula (I):

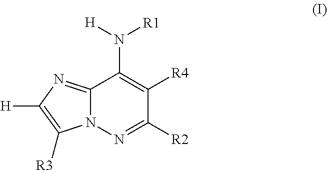

These compounds are inhibitors of CDK9 and are useful in the treatment of a variety of diseases including hyperproliferative diseases, virally induced infectious diseases, and cardiovascular diseases.

The present invention relates to the field of biomedicine, and specifically includes a series of novel CDK9 inhibitors with imidazopyridazine structure and their uses. It includes compounds as CDK9 inhibitors, pharmaceutically acceptable salts, solvates of the above compounds or prodrugs, and the use of pharmaceutical compositions of the above-mentioned compounds.

Cyclin-dependent kinases (CDKs) are a member of the serine/threonine kinase family and form a heterodimer with the regulatory subunit cyclin to perform its catalytic function. The members of the CDKs family can be divided into periodic CDKs and transcriptional CDKs according to their functional differences. The former mainly includes CDK1/2/4/6, which control the cell cycle process; the latter mainly includes CDK7/8/9, which regulate the transcription of mRNA and processing (Malumbres M. et al. Genome Biol., 2014, 15:122). Overexpression or functional enhancement of transcriptional CDKs will cause a significant increase in the expression of specific downstream genes, especially the anti-apoptotic protein Mcl-1, which will cause tumors (Morales F. et al. Cell Cycle, 2016, 15:519)-27). In recent years, it has been discovered that non-selective CDK inhibitors can achieve anti-tumor effects by inhibiting the function of CDK9. Therefore, the research on CDK inhibitors has attracted people's attention (Krystof V. et al. Target, Curr. Pharm. Des., 2012, 18:2883-2890). Studies have revealed that the overexpression of CDK9 is related to the occurrence of a variety of tumors, inflammation, and virus replication, such as acute myeloid leukemia, breast cancer, colon cancer, and prostate cancer, as well as human immunodeficiency virus and adenovirus (Franco L. C. et al. J. Cell Biochem., 2017, 119:1273-1284). These findings indicate that CDK9 is an effective target for cancer treatment. First generation pan-CDK inhibitors that have entered the clinic include Flavopiridol, Dinaciclib, SNS-032 and CYC065, but showed clinical side-effects due to their lack of selectivity over a range of CDKs. In recent years pharmaceutical research is focusing on the identification and development of mainly selective CDK9 inhibitors. These are currently in preclinical and early clinical research such as BAY-1143572, KB-0742, and AZD4573.

It has been an object of the present invention to provide for novel inhibitors of CDK9 which overcome the drawbacks of the compounds of the prior art. It was especially an object to increase selectivity to CDK9 and improve druggability. The main object of the present invention was to provide for a class of CDK9 small molecule inhibitors which show high efficiency, low toxicity, and excellent drug metabolism properties, and which can be used for disease prevention and/or treatment. This object has been solved by the compounds of the present invention. The present invention provides CDK9 small molecule inhibitors containing imidazopyridazine structures.

The present invention provides one or more compounds of formula (I):

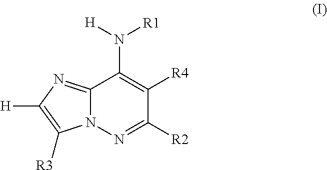

wherein
R1 is an alkyl, alkenyl, alkynyl, heteroalkyl, cycloalkyl, heterocycloalkyl, alkylcycloalkyl, heteroalkylcycloalkyl, aryl, heteroaryl, aralkyl or heteroaralkyl group; all of which groups may optionally be substituted;
R2 is an optionally substituted phenyl group;
R3 is a halogen atom;
R4 is a hydrogen atom or a halogen atom;
   or a salt thereof.

Preferably, R4 is H or Cl.

Moreover preferably, R4 is H.

Further preferably, R3 is Cl

Further preferably, R2 is a phenyl group that is substituted by 1, 2 or 3 substituents that are independently selected from F, Cl, Br and Me.

Moreover preferably, R2 is a phenyl group that is substituted in the 2 and 6 position (i.e., a phenyl group carrying two ortho substituents).

Most preferably, R2 is a 2,6-difluorophenyl group.

Preferably, R1 is an optionally substituted C₃₋₇ cycloalkyl group, an optionally substituted heterocycloalkyl group containing from 3 to 9 ring atoms that are independently selected from C, N and O, an optionally substituted phenyl group, an optionally substituted benzyl group, an optionally substituted heteroaryl group containing 5 or 6 ring atoms that are independently selected from C, N, O and S, or a group of formula —CH₂-Het, wherein Het is an optionally substituted heteroaryl group containing 5 or 6 ring atoms that are independently selected from C, N, O and S.

Further preferably, R1 is an optionally substituted C₃₋₇ cycloalkyl group, an optionally substituted heterocycloalkyl group containing from 3 to 9 ring atoms that are independently selected from C, N and O, or a group of formula —CH₂-Het, wherein Het is an optionally substituted heteroaryl group containing 5 or 6 ring atoms that are independently selected from C, N, O and S.

Moreover preferably, R1 is selected from the following groups:

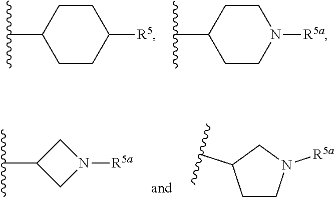

wherein R⁵ is NH₂, a C₁₋₈ heteroalkyl group, or an optionally substituted heterocycloalkyl group containing 5 or 6 ring atoms that are independently selected from C, N and O; and R^{5a} is hydrogen, a C₁₋₆ alkyl group, a C₁₋₈ heteroalkyl group, an optionally substituted C₅₋₆ cycloalkyl group or an optionally substituted heterocycloalkyl group containing 5 or 6 ring atoms that are independently selected from C, N and O.

Further preferably, R1 is selected from the following groups:

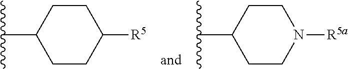

wherein R⁵ is NH₂, a C₁₋₆ heteroalkyl group, or an optionally substituted heterocycloalkyl group containing 5 or 6 ring atoms that are independently selected from C, N and O; and R^{5a} is hydrogen, a C₁₋₆ alkyl group, an optionally substituted C₅₋₆ cycloalkyl group or an optionally substituted heterocycloalkyl group containing 5 or 6 ring atoms that are independently selected from C, N and 0.

Moreover preferably, R^{5a} is hydrogen, a C₁₋₆ alkyl group, or a group of formula —SO₂—C₁₋₆ alkyl, —CONH—C₁₋₆ alkyl, or —COO—C₁₋₆ alkyl; or an optionally substituted C₅₋₆ cycloalkyl group or an optionally substituted heterocycloalkyl group containing 5 or 6 ring atoms that are independently selected from C, N and O.

Further preferably, R⁵ is a group of formula —N(CH₂CH₂)₂NMe, NH₂, —N(CH₂CH₂)₂O, or —NMe₂.

Further preferably, R^{5a} is a group of formula -Me, —SO₂Me, or —COO^{t}Bu.

Moreover preferably, R1 is a group of formula —CH₂-Het wherein Het is an optionally substituted pyridyl group.

Further preferably, the optional substituents at group R1 are independently selected from —N(CH₂CH₂)₂NMe, —N(CH₂CH₂)₂O, NH₂, —NMe₂, -Me, —SO₂Me, and —COO^{t}Bu.

Further preferably, R1 an optionally substituted heteroalkyl group containing from 1 to 12 carbon atoms and from 1 to 6 heteroatoms selected from N, O and S.

Further preferably, R1 is an optionally substituted C₄₋₆ cycloalkyl group, an optionally substituted heterocycloalkyl group containing from 5 to 8 ring atoms that are independently selected from C, N and O, an optionally substituted phenyl group or an optionally substituted pyrazol-4-yl group.

Moreover preferably, R1 is selected from the following groups:

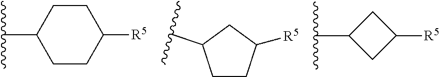

wherein R⁵ is a —NH₂ group, a C₁₋₆ heteroalkyl group or an optionally substituted heterocycloalkyl group containing 5 or 6 ring atoms that are independently selected from C, N and O.

Preferably, R5 is selected from the following groups: —NH₂, —NMe₂, pyrrolidinyl, piperidinyl, morpholinyl, —NH(CH₂)₂F, —NH(CH₂)₃F, —NH(CH₂)₂OH, —NH(CH₂)₃OH, —NH(CH₂)₂OMe, —NH(CH₂)₃OMe, —N(Me)(CH₂)₂F, —N(Me)(CH₂)₃F, —N(Me)(CH₂)₂OH, —N(Me)(CH₂)₃OH, —N(Me)(CH₂)₂OMe, and —N(Me)(CH₂)₃OMe.

Moreover preferably, R1 is selected from the following groups:

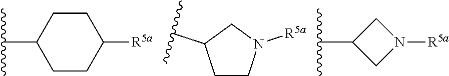

wherein R^{5a} is hydrogen, a C₁₋₆ alkyl group, a C₁₋₈ heteroalkyl group, an optionally substituted C₅₋₆ cycloalkyl group or an optionally substituted heterocycloalkyl group containing 5 or 6 ring atoms that are independently selected from C, N and O.

Further preferably, R1 has the following structure:

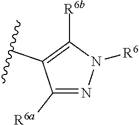

wherein R^{6a} is hydrogen, halogen or a C₁₋₄ heteroalkyl group (especially a —O—C₁₋₄ alkyl group); R^{6b} is hydrogen, halogen or a C₁₋₄ heteroalkyl group (especially a —O—C₁₋₄ alkyl group) and R⁶ is a C₁₋₆ alkyl group, a C₁₋₆ heteroalkyl group, an optionally substituted C₃₋₇ cycloalkyl group or an optionally substituted heterocycloalkyl group containing from 3 to 7 ring atoms that are independently selected from C, N and O.

Preferably, R^{6a} is hydrogen, Cl or OMe.

Moreover preferably, R^{6b} is hydrogen.

Further preferably, R⁶ is a methyl group, a group of formula —C(CH₃)₂CN, an optionally substituted cyclohexyl group, an optionally substituted piperidinyl group or a tetrahydropyranyl group.

Moreover preferably, R⁶ is a group of formula -Cy-L-R^{6c}, wherein Cy is an optionally substituted C₃₋₇ cycloalkylene group or an optionally substituted heterocycloalkylene group containing from 3 to 7 ring atoms that are independently selected from C, N and O; L is a bond or a CH₂ group and R^{6c} is an optionally substituted C₃₋₇ cycloalkyl group or an optionally substituted heterocycloalkyl group containing from 3 to 7 ring atoms that are independently selected from C, N and O.

Further preferably, Cy is a cyclohexylene group or a piperidinylene group.

Moreover preferably, R^{6c} is a cyclopropyl group or an optionally substituted heterocycloalkyl group containing from 4 to 6 ring atoms that are independently selected from C, N and O.

Further preferably, R1 has the following structure:

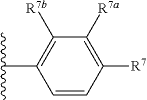

wherein R^{7a} is hydrogen, halogen, CN or an —O—C₁₋₄ alkyl group; R^{7b} is hydrogen, halogen, CN or an —O—C₁₋₄ alkyl group and R⁷ is a C₁₋₆ heteroalkyl group, an optionally substituted heterocycloalkyl group containing from 3 to 7 ring atoms that are independently selected from C, N and O or an optionally substituted heteroalkylcycloalkyl group containing from 4 to 12 atoms that are independently selected from C, N and O.

Preferably, R^{7b} is hydrogen or a methoxy group; preferably hydrogen.

Further preferably, R^{7a} is hydrogen, fluorine, CN or an —O—C₁₋₄ alkyl group; preferably hydrogen.

Moreover preferably, R⁷ is an optionally substituted piperazinyl group, an optionally substituted piperidinyl group, an optionally substituted morpholinyl group or an optionally substituted tetrahydropyridinyl group.

Further preferably, R⁷ is a group of formula -Cy′-L′-R^{7c}, wherein Cy′ is an optionally substituted C₃₋₇ cycloalkylene group or an optionally substituted heterocycloalkylene group containing from 3 to 7 ring atoms that are independently selected from C, N and O; L′ is a bond or a CH₂ group and R^{7c} is an optionally substituted C₃₋₇ cycloalkyl group or an optionally substituted heterocycloalkyl group containing from 3 to 7 ring atoms that are independently selected from C, N and O.

Moreover preferably, R⁷ is a group of formula —CO—R^{7d} or —CO—NH—R^{7d}, wherein R^{7d} is a C₁₋₄ alkyl group or an optionally substituted heterocycloalkylene group containing from 3 to 7 ring atoms that are independently selected from C, N and O.

Further preferably, R1 is a group of formula —CH₂—R^{1a} or —CH₂—CH₂—R^{1a}, wherein R^{1a} is an optionally substituted heterocycloalkyl group having 5 or 6 ring atoms that are independently selected from C, N and O (Preferably, the optional substituent at R^{1a} is a C₁₋₄ alkyl group).

The most preferred compounds of the present invention are the compounds disclosed in the examples, or a salt thereof.

It is further preferred to combine the preferred embodiments of the present invention in any desired manner (e.g., any embodiment for R1 may be combined with any embodiment of R2).

The expression alkyl refers to a saturated, straight-chain or branched hydrocarbon group that contains from 1 to 20 carbon atoms, preferably from 1 to 10 carbon atoms, especially from 1 to 6 (e.g. 1, 2, 3 or 4) carbon atoms, for example a methyl (Me, CH₃), ethyl (Et), n-propyl (nPr), iso-propyl (iPr), n-butyl (nBu), iso-butyl (iBu), sec-butyl (sBu), tert-butyl (tBu), n-pentyl, iso-pentyl, n-hexyl, 2,2-dimethylbutyl or n-octyl group.

The expression C₁₋₆ alkyl refers to a saturated, straight-chain or branched hydrocarbon group that contains from 1 to 6 carbon atoms. The expression C₁₋₄ alkyl refers to a saturated, straight-chain or branched hydrocarbon group that contains from 1 to 4 carbon atoms. Examples are a methyl, ethyl, n-propyl, iso-propyl, n-butyl, iso-butyl, sec-butyl or tert-butyl group.

The expressions alkenyl and alkynyl refer to at least partially unsaturated, straight-chain or branched hydrocarbon groups that contain from 2 to 20 carbon atoms, preferably from 2 to 15 carbon atoms, especially from 2 to 10 (e.g., 2, 3 or 4) carbon atoms, for example an ethenyl (vinyl), propenyl (allyl), iso-propenyl, butenyl, ethynyl (acetylenyl), propynyl (e.g., propargyl), butynyl, isoprenyl or hex-2-enyl group. Preferably, alkenyl groups have one or two (especially preferably one) double bond(s), and alkynyl groups have one or two (especially preferably one) triple bond(s).

Furthermore, the terms alkyl, alkenyl and alkynyl refer to groups in which one or more hydrogen atoms have been replaced by a halogen atom (preferably F or Cl) such as, for example, a 2,2,2-trichloroethyl or a trifluoromethyl group.

The expression heteroalkyl refers to an alkyl, alkenyl or alkynyl group in which one or more (preferably 1 to 8; especially preferably 1, 2, 3 or 4) carbon atoms have been replaced by an oxygen, nitrogen, phosphorus, boron, selenium, silicon or sulfur atom (preferably by an oxygen, sulfur or nitrogen atom) or by a SO or a SO₂ group. The expression heteroalkyl furthermore refers to a carboxylic acid or to a group derived from a carboxylic acid, such as, for example, acyl, acylalkyl, alkoxycarbonyl, acyloxy, acyloxyalkyl, carboxyalkylamide or alkoxycarbonyloxy. Furthermore, the term heteroalkyl refers to groups in which one or more hydrogen atoms have been replaced by a halogen atom (preferably F or Cl).

Preferably, a heteroalkyl group contains from 1 to 12 carbon atoms and from 1 to 8 heteroatoms selected from oxygen, nitrogen and sulfur (especially oxygen and nitrogen). Especially preferably, a heteroalkyl group contains from 1 to 6 (e.g. 1, 2, 3 or 4) carbon atoms and 1, 2, 3 or 4 (especially 1, 2 or 3) heteroatoms selected from oxygen, nitrogen and sulfur (especially oxygen and nitrogen). The term C₁₋₈ heteroalkyl refers to a heteroalkyl group containing from 1 to 8 carbon atoms and 1, 2, 3, 4 or 5 heteroatoms selected from O, S and/or N (especially O and/or N). The term C₁₋₆ heteroalkyl refers to a heteroalkyl group containing from 1 to 6 carbon atoms and 1, 2, 3 or 4 heteroatoms selected from O, S and/or N (especially O and/or N). The term C₁₋₄ heteroalkyl refers to a heteroalkyl group containing from 1 to 4 carbon atoms and 1, 2 or 3 heteroatoms selected from O, S and/or N (especially O and/or N).

Examples of heteroalkyl groups are groups of formulae: R^{a}—O—Y^{a}—, R^{a}—S—Y^{a}—, R^{a}—SO—Y^{a}—, R^{a}—SO₂—Y^{a}—, R^{a}—N(R^{b})—SO₂—Y^{a}—, R^{a}—SO₂—N(R^{b})—Y^{a}—, R^{a}—N(R^{b})—Y^{a}—, R^{a}—CO—Y^{a}—, R^{a}—O—CO—Y^{a}—, R^{a}—CO—O—Y^{a}—, R^{a}—CO—N(R^{b})—Y^{a}—, R^{a}—N(R^{b})—CO—Y^{a}—, R^{a}—O—CO—N(R^{b})—Y^{a}—, R^{a}—N(R^{b})—CO—O—Y^{a}—, —Y^{a}—CN, R^{a}—N(R^{b})—CO—N(R^{c})—Y^{a}—, R^{a}—O—CO—O—Y^{a}—, R^{a}—N(R^{b})—C(═NR^{d})—N(R^{c})—Y^{a}—, R^{a}—CS—Y^{a}—, R^{a}—O—CS—Y^{a}—, R^{a}—CS—O—Y^{a}—, R^{a}—CS—N(R^{b})—Y^{a}—, R^{a}—N(R^{b})—CS—Y^{a}—, R^{a}—O—CS—N(R^{b})—Y^{a}—, R^{a}—N(R^{b})—CS—O—Y^{a}—, R^{a}—N(R^{b})—CS—N(R^{c})—Y^{a}—, R^{a}—O—CS—O—Y^{a}—, R^{a}—S—CO—Y^{a}—, R^{a}—CO—S—Y^{a}—, R^{a}—S—CO—N(R^{b})—Y^{a}—, R^{a}—N(R^{b})—CO—S—Y^{a}—, R^{a}—S—CO—O—Y^{a}—, R^{a}—O—CO—S—Y^{a}—, R^{a}—S—CO—S—Y^{a}—, R^{a}—S—CS—Y^{a}—, R^{a}—CS—S—Y^{a}—, R^{a}—S—CS—N(R^{b})—Y^{a}—, R^{a}—N(R^{b})—CS—S—Y^{a}—, R^{a}—S—CS—O—Y^{a}—, R^{a}—O—CS—S—Y^{a}—, wherein R^{a} being a hydrogen atom, a C₁-C₆ alkyl, a C₂-C₆ alkenyl or a C₂-C₆ alkynyl group; R^{b} being a hydrogen atom, a C₁-C₆ alkyl, a C₂-C₆ alkenyl or a C₂-C₆ alkynyl group; R^{c} being a hydrogen atom, a C₁-C₆ alkyl, a C₂-C₆ alkenyl or a C₂-C₆ alkynyl group; Rd being a hydrogen atom, a C₁-C₆ alkyl, a C₂-C₆ alkenyl or a C₂-C₆ alkynyl group and Y^{a} being a bond, a C₁-C₆ alkylene, a C₂-C₆ alkenylene or a C₂-C₆ alkynylene group, wherein each heteroalkyl group contains at least one carbon atom and one or more hydrogen atoms may be replaced by fluorine or chlorine atoms.

Specific examples of heteroalkyl groups are methoxy, trifluoromethoxy, ethoxy, n-propyloxy, iso-propyloxy, n-butoxy, tert-butyloxy, methoxymethyl, —CH₂CH₂OH, —CH₂OH, —SO₂Me, —NHAc, —C(CH₃)₂CN, —COO^{t}Bu, methoxyethyl, ethoxymethyl, 1-methoxyethyl, 1-ethoxyethyl, 2-methoxyethyl or 2-ethoxyethyl, methylamino, ethylamino, propylamino, isopropylamino, dimethylamino, diethylamino, isopropylethylamino, methylamino methyl, ethylamino methyl, diisopropylamino ethyl, methylthio, ethylthio, isopropylthio, enol ether, dimethylamino methyl, dimethylamino ethyl, acetyl, propionyl, butyryloxy, acetyloxy, methoxycarbonyl, ethoxycarbonyl, propionyloxy, acetylamino or propionylamino, carboxymethyl, carboxyethyl or carboxypropyl, N-ethyl-N-methylcarbamoyl or N-methylcarbamoyl. Further examples of heteroalkyl groups are nitrile (—CN), isonitrile, cyanate, thiocyanate, isocyanate, isothiocyanate and alkylnitrile groups.

The expression cycloalkyl refers to a saturated or partially unsaturated (for example, a cycloalkenyl group) cyclic group that contains one or more rings (preferably 1 or 2), and contains from 3 to 14 ring carbon atoms, preferably from 3 to 10 (especially 3, 4, 5, 6 or 7) ring carbon atoms. The expression cycloalkyl refers furthermore to groups in which one or more hydrogen atoms have been replaced by fluorine, chlorine, bromine or iodine atoms or by OH, ═O, SH, ═S, NH₂, ═NH, N₃ or NO₂ groups, thus, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetraline, cyclopentylcyclohexyl, fluorocyclohexyl or cyclohex-2-enyl group. Preferably, the expression cycloalkyl refers to a saturated cyclic group that contains one or more rings (preferably 1 or 2), and contains from 3 to 14 ring carbon atoms, preferably from 3 to 10 (especially 3, 4, 5, 6 or 7) ring carbon atoms.

The expression heterocycloalkyl refers to a cycloalkyl group as defined above in which one or more (preferably 1, 2 or 3) ring carbon atoms have been replaced by an oxygen, nitrogen, silicon, selenium, phosphorus or sulfur atom (preferably by an oxygen, sulfur or nitrogen atom) or a SO group or a SO₂ group. A heterocycloalkyl group has preferably 1 or 2 ring(s) and 3 to 10 (especially 3, 4, 5, 6 or 7) ring atoms (preferably selected from C, O, N and S). The expression heterocycloalkyl refers furthermore to groups that are substituted by fluorine, chlorine, bromine or iodine atoms or by OH, ═O, SH, ═S, NH₂, —NH, N₃ or NO₂ groups. Examples are a piperidyl, prolinyl, imidazolidinyl, piperazinyl, morpholinyl (e.g. —N(CH₂CH₂)₂O), urotropinyl, pyrrolidinyl, tetrahydrothiophenyl, tetrahydropyranyl, tetrahydrofuryl or 2-pyrazolinyl group and also lactames, lactones, cyclic imides and cyclic anhydrides.

The expression alkylcycloalkyl refers to groups that contain both cycloalkyl and alkyl, alkenyl or alkynyl groups in accordance with the above definitions, for example alkylcycloalkyl, cycloalkylalkyl, alkylcycloalkenyl, alkenylcycloalkyl and alkynylcycloalkyl groups. An alkylcycloalkyl group preferably contains a cycloalkyl group that contains one or two rings and from 3 to 10 (especially 3, 4, 5, 6 or 7) ring carbon atoms, and one or two alkyl, alkenyl or alkynyl groups (especially alkyl groups) having 1 or 2 to 6 carbon atoms.

The expression heteroalkylcycloalkyl refers to alkylcycloalkyl groups as defined above in which one or more (preferably 1, 2 or 3) carbon atoms have been replaced by an oxygen, nitrogen, silicon, selenium, phosphorus or sulfur atom (preferably by an oxygen, sulfur or nitrogen atom) or a SO group or a SO₂ group. A heteroalkylcycloalkyl group preferably contains 1 or 2 rings having from 3 to 10 (especially 3, 4, 5, 6 or 7) ring atoms, and one or two alkyl, alkenyl, alkynyl or heteroalkyl groups (especially alkyl or heteroalkyl groups) having from 1 or 2 to 6 carbon atoms. Examples of such groups are alkylheterocycloalkyl, alkylheterocycloalkenyl, alkenylheterocycloalkyl, alkynylheterocycloalkyl, heteroalkylcycloalkyl, heteroalkylheterocycloalkyl and heteroalkylheterocycloalkenyl, the cyclic groups being saturated or mono-, di- or tri-unsaturated.

The expression aryl refers to an aromatic group that contains one or more rings and from 6 to 14 ring carbon atoms, preferably from 6 to 10 (especially 6) ring carbon atoms. The expression aryl refers furthermore to groups that are substituted by fluorine, chlorine, bromine or iodine atoms or by OH, SH, NH₂, N₃ or NO₂ groups. Examples are the phenyl (Ph), naphthyl, biphenyl, 2-fluorophenyl, anilinyl, 3-nitrophenyl or 4-hydroxyphenyl group.

The expression heteroaryl refers to an aromatic group that contains one or more rings and from 5 to 14 ring atoms, preferably from 5 to 10 (especially 5 or 6 or 9 or 10) ring atoms, comprising one or more (preferably 1, 2, 3 or 4) oxygen, nitrogen, phosphorus or sulfur ring atoms (preferably O, S or N). The expression heteroaryl refers furthermore to groups that are substituted by fluorine, chlorine, bromine or iodine atoms or by OH, SH, N₃, NH₂ or NO₂ groups. Examples are pyridyl (e.g. 4-pyridyl), imidazolyl (e.g. 2-imidazolyl), phenylpyrrolyl (e.g. 3-phenylpyrrolyl), thiazolyl, isothiazolyl, 1,2,3-triazolyl, 1,2,4-triazolyl, oxadiazolyl, thiadiazolyl, indolyl, indazolyl, tetrazolyl, pyrazinyl, pyrimidinyl, pyridazinyl, 4-hydroxypyridyl (4-pyridonyl), 3,4-hydroxypyridyl (3,4-pyridonyl), oxazolyl, isoxazolyl, triazolyl, tetrazolyl, isoxazolyl, indazolyl, indolyl, benzimidazolyl, benzoxazolyl, benzisoxazolyl, benzthiazolyl, pyridazinyl, quinolinyl, isoquinolinyl, pyrrolyl, purinyl, carbazolyl, acridinyl, pyrimidyl, 2,3′-bifuryl, pyrazolyl (e.g. 3-pyrazolyl) and isoquinolinyl groups.

The expression aralkyl refers to groups containing both aryl and also alkyl, alkenyl, alkynyl and/or cycloalkyl groups in accordance with the above definitions, such as, for example, arylalkyl, arylalkenyl, arylalkynyl, arylcycloalkyl, arylcycloalkenyl, alkylarylcycloalkyl and alkylarylcycloalkenyl groups. Specific examples of aralkyls are phenylcyclopentyl, cyclohexylphenyl as well as groups derived from toluene, xylene, mesitylene, styrene, benzyl chloride, o-fluorotoluene, 1H-indene, tetraline, dihydronaphthalene, indanone, cumene, fluorene and indane. An aralkyl group preferably contains one or two aromatic ring systems (especially 1 or 2 rings), each containing from 6 to 10 carbon atoms and one or two alkyl, alkenyl and/or alkynyl groups containing from 1 or 2 to 6 carbon atoms and/or one or two cycloalkyl group containing 3, 4, 5, 6 or 7 ring carbon atoms.

The expression heteroaralkyl refers to groups containing both aryl and/or heteroaryl groups and also alkyl, alkenyl, alkynyl and/or heteroalkyl and/or cycloalkyl and/or heterocycloalkyl groups in accordance with the above definitions. A heteroaralkyl group preferably contains one or two aromatic ring systems (especially 1 or 2 rings), each containing from 5 or 6 to 9 or 10 ring atoms (preferably selected from C, N, O and S) and one or two alkyl, alkenyl and/or alkynyl groups containing 1 or 2 to 6 carbon atoms and/or one or two heteroalkyl groups containing 1 to 6 carbon atoms and 1, 2 or 3 heteroatoms selected from O, S and N and/or one or two cycloalkyl groups each containing 3, 4, 5, 6 or 7 ring carbon atoms and/or one or two heterocycloalkyl groups, each containing 3, 4, 5, 6 or 7 ring atoms comprising 1, 2, 3 or 4 oxygen, sulfur or nitrogen atoms.

Examples are arylheteroalkyl, arylheterocycloalkyl, arylheterocycloalkenyl, arylalkylheterocycloalkyl, arylalkenylheterocycloalkyl, arylalkynylheterocycloalkyl, arylalkylheterocycloalkenyl, heteroarylalkyl, heteroarylalkenyl, heteroarylalkynyl, heteroarylheteroalkyl, heteroarylcycloalkyl, heteroarylcycloalkenyl, heteroaryl-heterocycloalkyl, heteroarylheterocycloalkenyl, heteroarylalkylcycloalkyl, heteroaryl-alkylheterocycloalkenyl, heteroarylheteroalkylcycloalkyl, heteroarylheteroalkyl-cycloalkenyl and heteroarylheteroalkylheterocycloalkyl groups, the cyclic groups being saturated or mono-, di- or tri-unsaturated. Specific examples are a tetrahydroisoquinolinyl, benzoyl, phthalidyl, 2- or 3-ethylindolyl, 4-methylpyridino, 2-, 3- or 4-methoxyphenyl, 4-ethoxyphenyl, 2-, 3- or 4-carboxyphenylalkyl group.

As already stated above, the expressions cycloalkyl, heterocycloalkyl, alkylcycloalkyl, heteroalkylcycloalkyl, aryl, heteroaryl, aralkyl and heteroaralkyl also refer to groups that are substituted by fluorine, chlorine, bromine or iodine atoms or by OH, ═O, SH, ═S, NH₂, ═NH, N₃ or NO₂ groups.

According to a preferred embodiment, all alkyl, alkenyl, alkynyl, heteroalkyl, cycloalkyl, heterocycloalkyl, alkylcycloalkyl, heteroalkylcycloalkyl, aryl, heteroaryl, aralkyl and heteroaralkyl groups described herein may optionally be substituted.

The term halogen refers to F, Cl, Br or I.

When an aryl, heteroaryl, cycloalkyl, alkylcycloalkyl, heteroalkylcycloalkyl, heterocycloalkyl, aralkyl or heteroaralkyl group contains more than one ring, these rings may be bonded to each other via a single or double bond or these rings may be annulated or fused or bridged.

The suffix “-ene” like e.g. in “phenylene” refers to the corresponding divalent group.

The term “optionally substituted” refers to a group which is unsubstituted or substituted by one or more (especially by one, two or three; preferably by one or two) substituents.

If a group comprises more than one substituent, these substituents are independently selected, i.e. they may be the same or different.

If a group is substituted by a cyclic group, such as e.g. a cycloalkyl group or a heterocycloalkyl group, this cyclic group may be bonded to said group via a single or double bond or this cyclic group may be annulated or fused to said group.

Specific examples for substituents are fluorine, chlorine, bromine and iodine and OH, SH, NH₂, —SO₃H, —SO₂NH₂, —COOH, —COOMe, —COMe (Ac), —NHSO₂Me, —SO₂NMe₂, —CH₂NH₂, —NHAc, —SO₂Me, —COO Bu, NMe₂, Me, —N(CH₂CH₂)₂NMe, —N(CH₂CH₂)₂O, —CONH₂, —CN, —NHCONH₂, —NHC(NH)NH₂, —NOHCH₃, —N₃ and —NO₂ groups.

Further examples of substituents are C₁-C₁₀ alkyl, C₂-C₁₀ alkenyl, C₂-C₁₀ alkynyl, C₁-C₁₀ heteroalkyl, C₃-C₁₈ cycloalkyl, C₁-C₁₇ heterocycloalkyl, C₄-C₂₀ alkylcycloalkyl, C₁-C₁₉ heteroalkylcycloalkyl, C₆-C₁₈ aryl, C₁-C₁₇ heteroaryl, C₇-C₂₀ aralkyl and C₁-C₁₉ heteroaralkyl groups; especially C₁-C₆ alkyl, C₂-C₆ alkenyl, C₂-C₆ alkynyl, C₁-C₆ heteroalkyl, C₃-C₁₀ cycloalkyl, C₁-C₉ heterocycloalkyl, C₄-C₁₂ alkylcycloalkyl, C₁-C₁₁ heteroalkylcycloalkyl, C₆-C₁₀ aryl, C₁-C₉ heteroaryl, C₇-C₁₂ aralkyl and C₁-C₁ heteroaralkyl groups, further preferably C₁-C₆ alkyl and C₁-C₆ heteroalkyl groups.

Preferred substituents are halogen atoms (e.g., F, Cl, Br) and —OH, —NH₂, —CN, C₁-C₆ alkyl, C₂-C₆ alkenyl, C₁-C₆ heteroalkyl, cyclopropyl and —CH₂-cyclopropyl groups.

Further preferred substituents are halogen atoms (e.g. F, Cl, Br) and —OH, —NH₂, —CN, —C₁₋₄ alkyl (e.g. -Me, -Et, -nPr, -iPr, -nBu, -iBu, -tBu, —CH₂CH₂F, CH₂CHF₂, —CH₂CF₃ and —CF₃), —O—C₁₋₄ alkyl (e.g. —OMe, —OEt, —O-nPr, —O-iPr, —O-nBu, —O-iBu and —O-tBu), —NHC₁₋₆ alkyl (e.g., —NH(CH₂)₂F and —NH(CH₂)₃F), —NH(CH₂)₂OH, —NH(CH₂)₃OH, —NH(CH₂)₂OMe, —NH(CH₂)₃OMe, —N(Me)(CH₂)₂OH, —N(Me)(CH₂)₃OH, —N(Me)(CH₂)₂OMe, —N(Me)(CH₂)₃OMe, —N(C₁₋₆ alkyl)₂, —C(CH₃)₂CN, —CONH—C₁₋₄ alkyl (e.g. —CONHCH₂CF₃, —CONHEt, —CONH^{t}Bu), —COOH, —COOMe, —COMe, —CH₂CH₂CH═CH₂, cyclopropyl and —CH₂-cyclopropyl groups.

The therapeutic use of compounds according to formula (I), their pharmacologically acceptable salts, solvates and hydrates, respectively, as well as formulations and pharmaceutical compositions also lie within the scope of the present invention.

The present invention further provides pharmaceutical compositions comprising one or more compounds of formula (I) or a salt thereof as defined herein or a pharmaceutically acceptable ester, prodrug, hydrate or solvate thereof, optionally in combination with a pharmaceutically acceptable carrier and/or adjuvant.

It is a further object of the present invention to provide a compound of formula (I) as defined herein or a pharmaceutical composition as defined herein for the preparation of a medicament for the treatment of one or more diseases specified herein.

Preferably the compounds of the present invention may be used for the treatment and/or prevention of the following conditions:

Diseases in which abnormal CDK (especially CDK9 and/or CDK2) regulation is observed, including broad number of cytokine-induced inflammatory, and autoimmune diseases, local or systemic viral infection diseases, viral infections of the eye, viral respiratory infections, or viral infections of the central and/or peripheral nervous system caused by DNA and/or RNA viruses, and various non-solid and solid malignancies, cancers, or hyperproliferative diseases such as acute myelogenous leukemia, chronic lymphocytic leukemia, relapsed multiple myeloma, non-Hodgkin's lymphoma, acute lymphoblastic leukemia, acute biphenotypic leukemia, aggressive MYC-driven B-Cell lymphoma, primary peritoneal carcinoma, Kaposi sarcoma, advanced breast cancer, non-small cell lung cancer, colorectal cancer, or liver cancer such as hepatocellular carcinoma, cervical intraepithelial neoplasia, prostate cancer, melanoma, glioma, glioblastoma, neuroblastoma, astrocytoma, anaplastic astrocytoma or glioblastoma including advanced and/or metastatic haematological/solid malignancies. In particular, the compounds may be used to treat a hematologic malignancy, or a solid tumor caused by aberrant expression of MYC- or MCL-1. Furthermore, the compounds may be used as modulator of the immune response and for the treatment and/or prevention of mechanical/injury-induced inflammation such as post-traumatic osteoarthritis (PTOA), systemic and local cytokine-induced inflammatory disease, including gastrointestinal or urinary tracts inflammatory diseases inflammatory diseases and inflammatory diseases of the eye, such as Sjogren's disease and glaucoma, bacteria-induced inflammatory disease, such as gingivitis, periodontitis, and for the treatment and/or prevention of cardiovascular diseases such as cardiac hypertrophy, dilated cardiomyopathy, atherosclerosis, and cardio-metabolic diseases such as obesity and diabetes.

A therapeutically effective amount of a compound in accordance with this invention means an amount of compound that is effective to prevent, alleviate or ameliorate symptoms of a disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage may be adjusted to the individual requirements in each particular case including the specific compound being administered, the route of administration, the condition being treated, as well as the patient being treated.

The salt of a compound of formula (I) is preferably a pharmacologically acceptable salt. Examples of pharmacologically acceptable salts of sufficiently basic compounds of formula (I) are salts of physiologically acceptable mineral acids like hydrochloric, hydrobromic, sulfuric and phosphoric acid; or salts of organic acids like methanesulfonic, p-toluenesulfonic, lactic, acetic, trifluoroacetic, citric, succinic, fumaric, maleic and salicylic acid. Further, a sufficiently acidic compound of formula (I) may form alkali or earth alkali metal salts, for example sodium, potassium, lithium, calcium or magnesium salts; ammonium salts; or organic base salts, for example methylamine, dimethylamine, trimethylamine, triethylamine, ethylenediamine, ethanolamine, choline hydroxide, meglumin, piperidine, morpholine, tris-(2-hydroxyethyl)amine, lysine or arginine salts; all of which are also further examples of salts of formula (I).

Compounds of formula (I) may be solvated, especially hydrated. The hydratization/hydration may occur during the process of production or as a consequence of the hygroscopic nature of the initially water free compounds of formula (I). The solvates and/or hydrates may e.g. be present in solid or liquid form.

It should be appreciated that certain compounds of formula (I) may have tautomeric forms from which only one might be specifically mentioned or depicted in the following description, different geometrical isomers (which are usually denoted as cis/trans isomers or more generally as (E) and (Z) isomers) or different optical isomers as a result of one or more chiral carbon atoms (which are usually nomenclatured under the Cahn-Ingold-Prelog or R/S system). All these tautomeric forms, geometrical or optical isomers (as well as racemates and diastereomers) and polymorphous forms are included in the invention. Since the compounds of formula (I) may contain asymmetric C-atoms, they may be present either as achiral compounds, mixtures of diastereomers, mixtures of enantiomers or as optically pure compounds. The present invention comprises both all pure enantiomers and all pure diastereomers, and also the mixtures thereof in any mixing ratio.

According to a further embodiment of the present invention, one or more hydrogen atoms of the compounds of the present invention may be replaced by deuterium. Deuterium modification improves the metabolic properties of a drug with little or no change in its intrinsic pharmacology. Deuterium substitution at specific molecular positions improves metabolic stability, reduces formation of toxic metabolites and/or increases the formation of desired active metabolites. Accordingly, the present invention also encompasses the partially and fully deuterated compounds of formula (I). The term hydrogen also encompasses deuterium.

The present invention also relates to pro-drugs which are composed of a compound of formula (I) and at least one pharmacologically acceptable protective group which will be cleaved off under physiological conditions, such as an alkoxy-, arylalkyloxy-, acyl-, acyloxymethyl group (e.g. pivaloyloxymethyl), an 2-alkyl-, 2-aryl- or 2-arylalkyl-oxycarbonyl-2-alkylidene ethyl group or an acyloxy group as defined herein, e.g. ethoxy, benzyloxy, acetyl or acetyloxy or, especially for a compound of formula (I), carrying a hydroxy group (—OH): a sulfate, a phosphate (—OPO3 or —OCH2OPO3) or an ester of an amino acid. Especially preferred are pro-drugs of the hydroxy group of a compound of formula (I).

As used herein, the term pharmaceutically acceptable ester especially refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

Preferably, the present invention also relates to a prodrug, a biohydrolyzable ester, a biohydrolyzable amide, a polymorph, tautomer, stereoisomer, metabolite, N-oxide, biohydrolyzable carbamate, biohydrolyzable ether, physiologically functional derivative, atropisomer, or in vivo-hydrolysable precursor, diastereomer or mixture of diastereomers, chemically protected form, affinity reagent, complex, chelate and a stereoisomer of the compounds of formula (I).

As mentioned above, therapeutically useful agents that contain compounds of formula (I), their solvates, salts or formulations are also comprised in the scope of the present invention. In general, compounds of formula (I) will be administered by using the known and acceptable modes known in the art, either alone or in combination with any other therapeutic agent.

For oral administration such therapeutically useful agents can be administered by one of the following routes: oral, e.g. as tablets, dragees, coated tablets, pills, semisolids, soft or hard capsules, for example soft and hard gelatine capsules, aqueous or oily solutions, emulsions, suspensions or syrups, parenteral including intravenous, intramuscular and subcutaneous injection, e.g. as an injectable solution or suspension, rectal as suppositories, by inhalation or insufflation, e.g. as a powder formulation, as microcrystals or as a spray (e.g. liquid aerosol), transdermal, for example via an transdermal delivery system (TDS) such as a plaster containing the active ingredient or intranasal. For the production of such tablets, pills, semisolids, coated tablets, dragees and hard, e.g. gelatine, capsules the therapeutically useful product may be mixed with pharmaceutically inert, inorganic or organic excipients as are e.g. lactose, sucrose, glucose, gelatine, malt, silica gel, starch or derivatives thereof, talc, stearinic acid or their salts, dried skim milk, and the like. For the production of soft capsules, one may use excipients as are e.g. vegetable, petroleum, animal or synthetic oils, wax, fat, polyols. For the production of liquid solutions, emulsions or suspensions or syrups one may use as excipients e.g. water, alcohols, aqueous saline, aqueous dextrose, polyols, glycerin, lipids, phospholipids, cyclodextrins, vegetable, petroleum, animal or synthetic oils. Especially preferred are lipids and more preferred are phospholipids (preferred of natural origin; especially preferred with a particle size between 300 to 350 nm) preferred in phosphate buffered saline (pH=7 to 8, preferred 7.4). For suppositories one may use excipients as are e.g. vegetable, petroleum, animal or synthetic oils, wax, fat and polyols. For aerosol formulations one may use compressed gases suitable for this purpose, as are e.g. oxygen, nitrogen and carbon dioxide. The pharmaceutically useful agents may also contain additives for conservation, stabilization, e.g. UV stabilizers, emulsifiers, sweetener, aromatizers, salts to change the osmotic pressure, buffers, coating additives and antioxidants.

In general, in the case of oral or parenteral administration to adult humans weighing approximately 80 kg, a daily dosage of about 10 mg to about 10,000 mg, preferably from about 20 mg to about 1,000 mg, should be appropriate, although the upper limit may be exceeded when indicated. The daily dosage can be administered as a single dose or in divided doses, or for parenteral administration, it may be given as continuous infusion or subcutaneous injection.

According to a moreover preferred embodiment, the present invention provides a method for treating one or more diseases specified herein which comprises administering to a subject in need of such treatment a therapeutically effective amount of a compound of formula (I), or a pharmaceutically acceptable salt thereof.

According to a further preferred embodiment, the present invention provides a method for treating one or more diseases specified herein which comprises administering to a subject in need of such treatment a pharmaceutical composition comprising a compound of formula (I), or a pharmaceutically acceptable salt thereof.

### EXAMPLES

### General Synthesis Methods

The following methods were used in synthesis of the compounds described herein.

Flash chromatography: Flash chromatography was performed on a Biotage Isolera® or Selekt® system using SNAP or SFÄR silica cartridges and ethyl acetate/cyclohexane/methanol or dichloromethane/methanol gradients as eluent.

Microwave conditions: Reactions under microwave conditions are performed in a Biotage initiator (R) microwave system.

SEMIprep reversed phase chromatography: The following instrumentation was used for SEMIprep reversed phase chromatography: 2× Varian PrepStar SD-1, 1× Dionex P580 Pump 1 Channel (MakeUP I), 1× Dionex AXP-MS (MakeUP II), 1× Dionex MSQ, 1× Dionex UVD 340V-Prep Flow Cell, Gilson 215 Liquid Handler, SunFire Prep C18 OBD 5 μm, 19×50 mm column, 1× G7159B 1290 Infinity II Preparative Open-Bed Sampler/Collector, 1× G7161B 1290 Infinity II Preparative Binary Pump, 1× G7111B 1260 Infinity II Quaternary Pump (Modifier), 1× G7111B 1260 Infinity II Quaternary Pump (Analyltic/MakeUp), 1× G7165A 1260 Infinity II Multiple Wavelength Detector incl. Flow Cell (Product #G1315-60022, Serial #DE185H6157, Path Length 10.00 mm, Volume 13.00 μl), 1× G7170B 1290 Infinity II MS Flow Modulator, 1× G6125B MSD 6100 Series Single Quadrupole incl. G1948B Electrospray Interface, and 3× G1170A 1290 Infinity Valve Drive (14 Ports, 6 Positions Valve Head for Analytic Column Selection; 14 Ports, 6 Positions Valve Head for Preparative Column Selection; 14 Ports, 2 Positions Valve Head for Analytic/Preparative Mode Selection).

Preparative columns: Waters SunFire Prep C18 5 μm OBD 30×100 mm, #186002572, Waters Atlantis T3 Prep 5 μm OBD 30×100 mm, #186003702, and Waters XSelect CSH Prep C18 5 μm OBD 30×100 mm, #186005425.

Analytical columns: Waters SunFire C18 2.5 μm 3.0×75 mm, #186005636, Waters Atlantis T3 3 μm 3.0×75 mm, #186005653, and Waters XSelect CSH C18 2.5 μm 3.0×75 mm, #186006106.

Typical chromatography conditions are as follows:

Column flow was 30 mL/min, Solvent A was methanol containing 0.3% acetic acid, and Solvent B was water containing 0.3% acetic acid.

Typical times and relative volumes of Solvent and Solvent B are shown in Table 1.

TABLE 1

| Time (min) | Solv. A | Solv. B |
|---|---|---|
| 0.0 | 30.00 | 70.00 |
| 10.0 | 100.00 | 0.00 |
| 14.0 | 100.00 | 0.00 |
| 14.4 | 30.00 | 70.00 |
| 16.4 | 30.00 | 70.00 |

Typical preparative method: Column flow was 60 mL/min, Solvent A was acetonitrile, and Solvent B was water. Preparation included Modifier Flow: 1.8 mL/min Modifier Flow containing 10% Acetic Acid in Acetonirile/Water 1:1=>resulting 0.3% Acetic Acid in Flow; and 0.5M NH₄Ac/NH₄OH-Buffer (pH 9.2) in Acetonitrile/Water 1:9=>resulting 15 mM Buffer Concentration in Flow.

MS MakeUp: 0.9 mL/min 0.05% Acetic Acid in Acetonirile/Water 1:1.

Typical Focused Gradient Timetable for e.g. 59.7% Elution Point is shown in Table 2.

TABLE 2

| Time (min) | Solv. A | Solv. B |
|---|---|---|
| −2.37 | 18.6 | 84.4 |
| 0.00 | 18.6 | 84.4 |
| 1.15 | 18.6 | 84.4 |
| 1.16 | 43.5 | 56.5 |
| 8.46 | 63.5 | 36.5 |
| 8.47 | 100 | 0 |
| 10.77 | 100 | 0 |
| 10.78 | 18.6 | 84.4 |

Typical Analytical Modifier: Column flow was 1 mL/min, Solvent A was acetonitrile, Solvent B was water, and Solvent C was 5% acetic Acid in acetonitrile/water 1:1.

Typical times and relative volumes of Solvent, Solvent B, Solvent C are shown in Table 3.

TABLE 3

| Time (min) | Solv. A | Solv. B | Solv. C |
|---|---|---|---|
| 0 | 2 | 96 | 2 |
| 0.5 | 2 | 96 | 2 |
| 5.5 | 96 | 2 | 2 |
| 5.6 | 98 | 0 | 2 |
| 6.9 | 98 | 0 | 2 |
| 7.0 | 2 | 2 | 2 |

A Mass Spectrometer Detector (API-ES, positive) at UV 220 nm, 254 nm, or 310 nm was used for detection.

Terms and abbreviations used in the Examples are provided in Table 4.

TABLE 4
Pd dppf - [1,1′-Bis(diphenylphosphino)ferrocene]dichloropalladium(II), complex with
dichloromethane CAS 95464-05-4
DCM - dichloromethane
THF- tetrahydofuran
MeOH- methanol
celite - Diatomaceous earth, celite (R) CAS 61790-53-2
diborolane - 4,4,5,5,4′,4′,5′,5′-Octamethyl-[2,2′]bi[[1,3,2]dioxaborolanyl] CAS 73183-34-3
pinacolborane - 4,4,5,5-Tetramethyl-[1,3,2]dioxaborolane CAS 25015-63-8
o.n. - over night
r.t. - room temperature
eq. - equivalent
dioxane - 1,4-dioxane
brine - saturated aqueous solution of NaCl
h - hour
TFA - trifluoroacetic acid
Boc - tert-Butyloxycarbonyl
catacxium- cataCXium ® A, Di(1-adamantyl)-n-butylphosphine, CAS 321921-71-5
LiHMDS - Lithium bis(trimethylsilyl)amide CAS 4039-32-1
Pd(PPh₃)₄ - Palladium-tetrakis(triphenylphosphine) CAS 14221-01-3
PYBOP - (Benzotriazol-1-yloxy)tripyrrolidinophosphonium hexafluorophosphate CAS
128625-52-5
DIPEA - N,N-Diisopropylethylamine CAS 7087-68-5
DMF - N,N-Dimethylformamide CAS 68-12-2
DBAD - Di-tert-butyl azodicarboxylate CAS 870-50-8
PPh₃ - Triphenylphosphine CAS 603-35-0
HOBT - 1-Hydroxybenzotriazole hydrate CAS 123333-53-9
HATU - 1-[Bis(dimethylamino)methylene]-1H-1,2,3-triazolo[4,5-b]pyridinium 3-oxide
hexafluorophosphate CAS 148893-10-1
EDCI - N-(3-Dimethylaminopropyl)-N′-ethylcarbodiimide hydrochloride CAS 25952-53-8
T3P - T3P ® 2,4,6-Tripropyl-1,3,5,2,4,6-trioxatriphosphorinane-2,4,6-trioxide CAS 68957-94-
8
NaOtBu Sodium tert-butoxide CAS 865-48-5
X-Phos Pd G2 - CAS 1310584-14-5
X-Phos - CAS 564483-18-7
tBuBrettPhos Pd G3 - CAS 1536473-72-9

The analytical HPLC Methods used in preparation of the compounds are listed in Table 5.

TABLE 5

| | | | flow | |
|---|---|---|---|---|
| Method | col | grad | (mL/min) | ms |
| HPLC-MS | Waters ACQUITY | 2%-4.0 min->95%-1.0 min->95%- | 0.6 | ESI positive |
| A | UPLC HSS T3 50 × 2.1 | 0.1 min->2%-2.9 min->2%; | | & negative |
| | 1.8 μm PN: 186003538 | ACN/H₂O + 0.1% HCOOH | | |
| HPLC-MS | Waters ACQUITY | 5%-4.0 min->95%-1.0 min->95%- | 0.6 | ESI positive |
| B | UPLC CSH C18 50 × 2.1 | 0.1 min->5%-2.9 min->5%; | | & negative |
| | 1.7 μm PN: 186005296 | ACN/H₂O + 0.1% HCOOH | | |
| HPLC-MS | Waters ACQUITY | 4%-4.0 min->96%-1.0 min->96%- | 0.6 | ESI positive |
| C | UPLC CSH C18 50 × 2.1 | 0.1 min->4%-2.9 min->4%; | | |
| | 1.7 μm PN: 186005296 | ACN/H₂O + 100 mM NH₄Ac- | | |
| HPLC-MS | Waters CORTECS | 5%-4.0 min->95%-1.0 min->95%- | 0.6 | ESI positive |
| D | UPLC C18+ 50 × 2.1 | 0.1 min->5%-2.9 min->5%; | | & negative |
| | 1.6 μm PN: 186007114 | ACN/H₂O + 0.1% HCOOH | | |
| HPLC-MS | Waters ACQUITY | 5%-0.75 min->95%-0.5 min->95%- | 1.2 | ESI positive |
| E | UPLC CSH C18 50 × 2.1 | 0.05 min->5%-1.2 min->5%; | | |
| | 1.7 μm PN: 186005296 | ACN/H₂O + 0.1% HCOOH | | |

### General Route A to Imidazopyridazines:

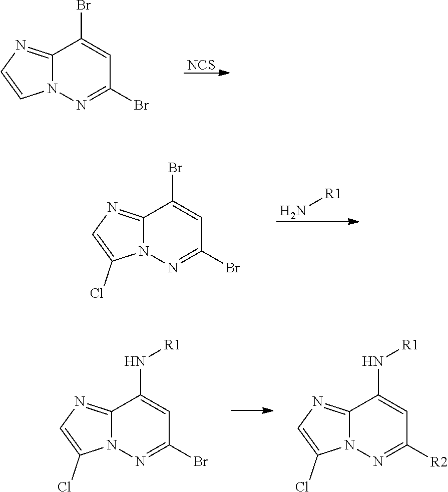

### Description of Synthesis Steps:

### Step 1: 6,8-dibromo-3-chloroimidazo[1,2-b]pyridazine

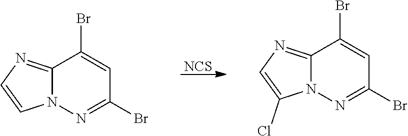 [SMILES: Brc1cc(Br)c2nccn2n1 ; "NCS" ; Clc1cnc2c(Br)cc(Br)nn12]

6,8-dibromoimidazo[1,2-b]pyridazine (1eq.) was dissolved in 1,2-dichloroathan and acetonitrile ia (1/1 v/v 16 mL/mmol), followed by the portion wise addition of NCS (1.3 eq.). The mixture was stirred at r.t. for 3 h. The mixture was quenched with sodium thiosulphate solution (5M in water), extracted with DCM, dried filtered and concentrated under reduced pressure. The crude product was purified by gradient flash chromatography.

### Step2: Amination

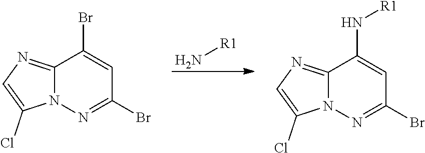

6,8-dibromo-3-chloroimidazo[1,2-b]pyridazine (1eq.) and the corresponding amine (1eq.) were dissolved in dioxane (3 mL/mmol). DIPEA (2.0 eq.) was added and the mixture heated to 180° C. for 30 min under microwave irritation. The mixture was diluted with NaHCO3 aq. sat. and extracted with DCM. The organic layer was dried (Na2SO4), filtered, and concentrated under reduced pressure. The crude product was purified by gradient flash chromatography.

### Step 3: Arylation

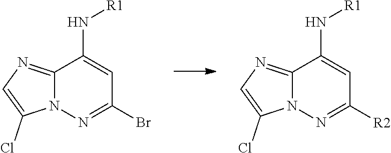

The product from the step before was dissolve in dioxane (0.5M). Water (10% v/v) was added. The corresponding boronic acid (1.3 eq.), Pd2(dba)3 (0.2 eq.), X-Phos (0.8 eq) and K2CO3 (3 eq.) were added. The mixture was degassed with N₂ and heated for one night at 80° C. Additional boronic acid, Pd2(dba)3 and X-Phos (same amounts than before) were added, followed by heating at 80° C. This was repeated until completion of the reaction (HPLC). The mixture was filtered through a plug of celite, washed with MeOH and water, concentrated, re-diluted with water and extracted with DCM. The organic layer was dried (Na₂SO₄), filtered and concentrated under reduced pressure. The crude material was purified by gradient flash chromatography, followed by preparative reversed phase HPLC.

The compounds of Examples #1, #3, #4, #10, #11, #12, #16, #17 #18 #19 were synthesized using these protocols.

The compounds of Example #14, #15 were synthesized using these protocols with modifications.

### Example #15

The compound of example #16 was dissolved in dry MeOH and treated with HCl (4M in dioxane) for 24 h. The mixture was concentrated under reduced pressure and the crude product purified by reversed phase HPLC.

### Example #14

The compound of example #15 was in DCM (dry), methane sulfonyl chloride (5 eq.) and triethylamine (5 eq.) were added. The mixture was stirred o.n. at r.t.. The mixture was dilute with water and extracted with EtOAc. The combined organic layers were dried filtered and concentrated under reduced pressure. The mixture was concentrated under reduced pressure and the crude product purified by reversed phase HPLC.

The compounds of examples #14, #15, #16, #18 are racemic mixtures.

### General Route B to Imidazopyridazines:

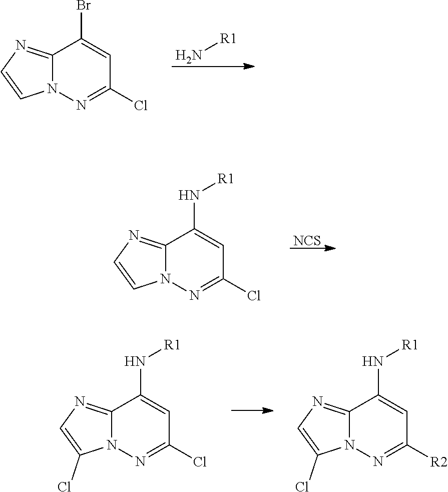

### Description of Synthesis Steps:

### Step 1: Amination

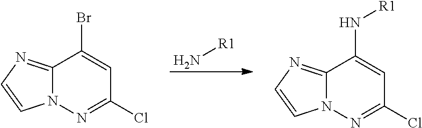

8-bromo-6-chloroimidazo[1,2-b]pyridazine (1eq.) and the corresponding amine as HCL salt (1.2 eq.) were mixed in dioxane (3 mL/mmol). DIPEA (6.0 eq.) was added and the mixture heated to 180° C. for 30 min under microwave irritation. The mixture was diluted with NaHCO3 aq. sat. and extracted with DCM. The organic layer was dried (Na2SO4), filtered, and concentrated under reduced pressure. The crude product was purified by gradient flash chromatography.

### Step 2: Chlorination

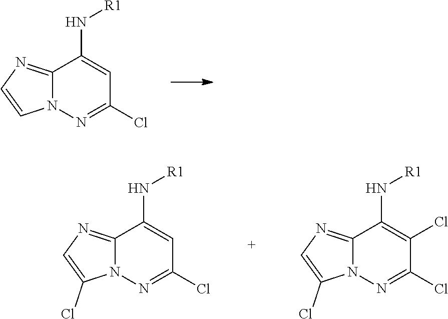

The product from the step before was dissolve in dioxane was dissolved in 1,2-dichloroethan and acetonitrile (1/1 v/v 16 mL/mmol), followed by the portion wise addition of NCS (1.3 eq.). The mixture was stirred at r.t. for 6 h, then at 40° C. o.n.. The reaction showed no completion (HPLC). Additional NCS (1eq.) was added. The mixture was heated for an additional night at 80° C. The two chlorinated products were observed. The mixture was quenched with sodium thiosulphate solution (5M in water), extracted with DCM, dried filtered and concentrated under reduced pressure. The crude product was purified by gradient flash chromatography. The two chloro intermediates were not separated.

### Step 3: Arylation

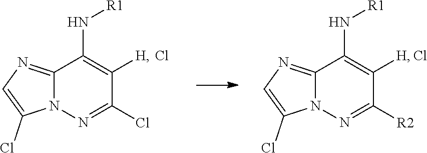

The product from the step before was dissolve in dioxane (0.5M). Water (10% v/v) was added. The corresponding boronic acid (1.3 eq.), Pd2(dba)3 (0.2 eq.), X-Phos (0.8 eq) and K2CO3 (3 eq.) were added. The mixture was degassed with N2 and heated for one night at 80° C. Additional boronic acid, Pd2(dba)3 and X-Phos (same amounts than before) were added, followed by heating at 80° C. This was repeated until completion of the reaction (HPLC). The mixture was filtered through a plug of celite, washed with MeOH and water, concentrated, re-diluted with water and extracted with DCM. The organic layer was dried (Na₂SO₄), filtered and concentrated under reduced pressure. The crude material was purified by gradient flash chromatography, followed by preparative reversed phase HPLC to separate the products from the mixture.

### General Route D to Imidazopyridazines:

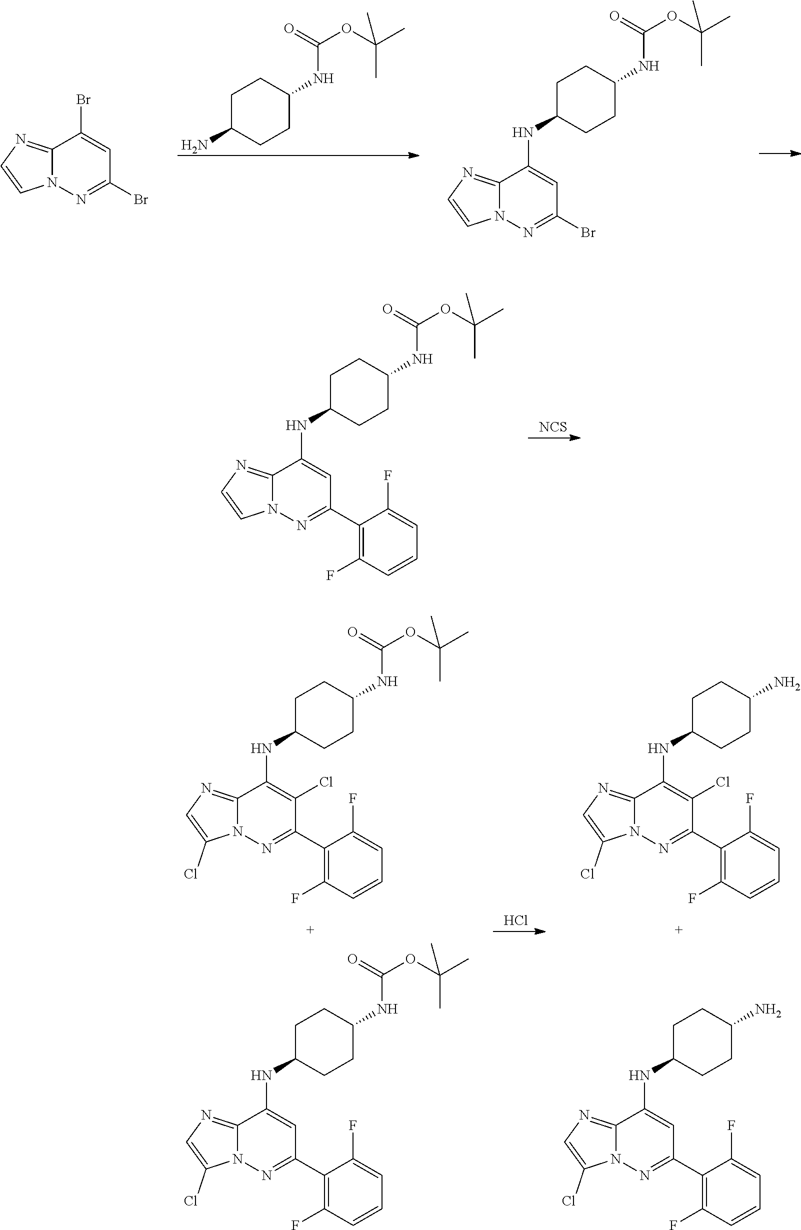

### Step1: Amination

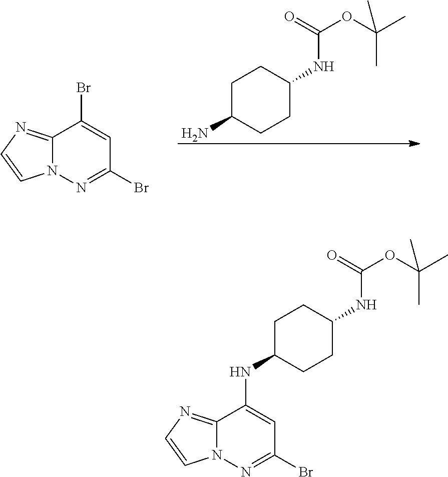 [SMILES: Brc1cc(Br)c2nccn2n1 ; CC(C)(C)OC(=O)N[C@H]1CC[C@H](N)CC1 ; CC(C)(C)OC(=O)N[C@H]1CC[C@H](Nc2cc(Br)nn3ccnc23)CC1]

6,8-dibromoimidazo[1,2-b]pyridazine (1eq.) and [4-(6-Bromo-imidazo[1,2-b]pyridazin-8-ylamino)-cyclohexyl]-carbamic acid tert-butyl ester as HCL salt (1.2 eq.) were mixed in dioxane (3 mL/mmol). DIPEA (6.0 eq.) was added and the mixture heated to 180° C. for 60 min under microwave irritation. The mixture was diluted with NaHCO3 aq. sat, and extracted with DCM. The organic layer was dried (Na2SO4), filtered, and concentrated under reduced pressure. The crude product was purified by gradient flash chromatography.

### Step2: Suzuki

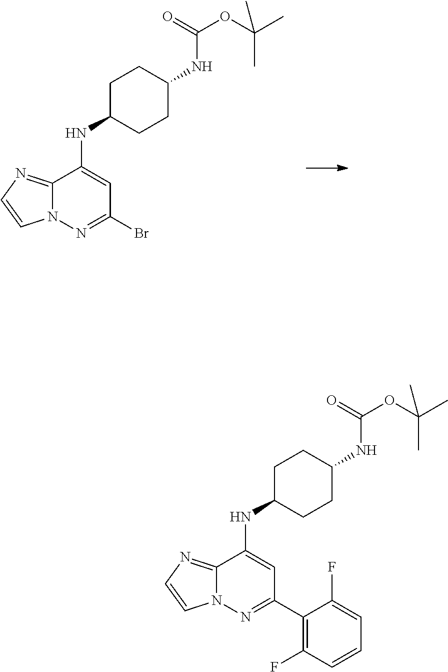 [SMILES: CC(C)(C)OC(=O)N[C@H]1CC[C@H](Nc2cc(Br)nn3ccnc23)CC1 ; CC(C)(C)OC(=O)N[C@H]1CC[C@H](Nc2cc(-c3c(F)cccc3F)nn3ccnc23)CC1]

The product from the step before was dissolve in dioxane (0.5M). Water (10% v/v) was added. 2,6-Di fluorophenyl boronic acid (1.3 eq.), Pd (dppf) DCM (0.2 eq.) and Na2CO3aq. (1M)(3 eq.) were added. The mixture was degassed with N2 and heated for 3 h at 80° C. Additional boronic acid and Pd(dppf)DCM were added, followed by heating at 80° C. This was repeated until completion of the reaction (HPLC). The mixture was filtered through a plug of celite, washed with MeOH and water, concentrated, re-diluted with water and extracted with DCM. The organic layer was dried (Na₂SO₄), filtered and concentrated under reduced pressure. The crude material was purified by gradient flash chromatography.

The product from the step before was dissolve in dioxane was dissolved in 1,2-dichloroethan and acetonitrile (1/1 v/v 10 mL/mmol), followed by the portion wise addition of NCS (1.3 eq.). The mixture was stirred at for 5 h at 80° C. The mixture was quenched with sodium thiosulphate solution (5M in water), extracted with DCM, dried filtered and concentrated under reduced pressure. The crude product was purified by gradient flash chromatography.

### Step 3: Chlorination

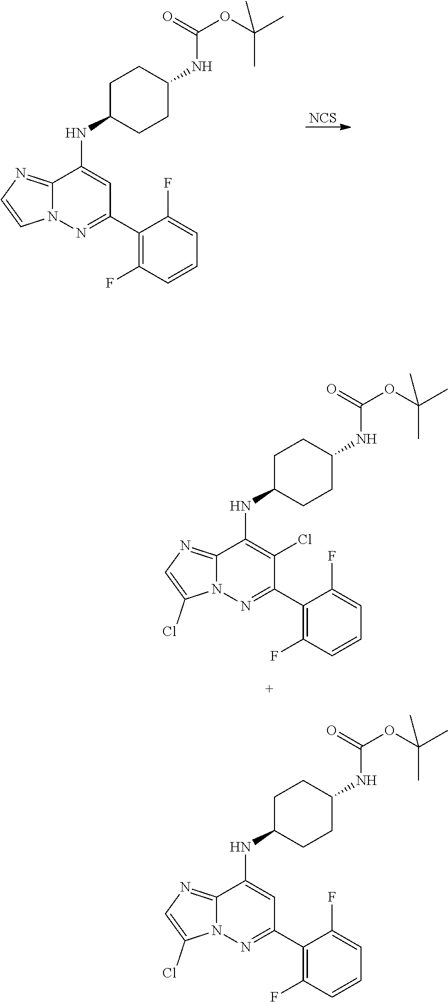 [SMILES: CC(C)(C)OC(=O)N[C@H]1CC[C@H](Nc2cc(-c3c(F)cccc3F)nn3ccnc23)CC1 ; "NCS" ; CC(C)(C)OC(=O)N[C@H]1CC[C@H](Nc2c(Cl)c(-c3c(F)cccc3F)nn3c(Cl)cnc23)CC1 ; CC(C)(C)OC(=O)N[C@H]1CC[C@H](Nc2cc(-c3c(F)cccc3F)nn3c(Cl)cnc23)CC1]

The product from the step before was dissolve in dioxane was dissolved in 1,2-dichloroethan and acetonitrile (1/1 v/v 10 mL/mmol), followed by the portion wise addition of NCS (1.3 eq.). The mixture was stirred at for 5 h at 60° C. The mixture was quenched with sodium thiosulphate solution (5M in water), extracted with DCM, dried filtered and concentrated under reduced pressure. The crude product mixture was used without purification.

### Step 4: Deprotection

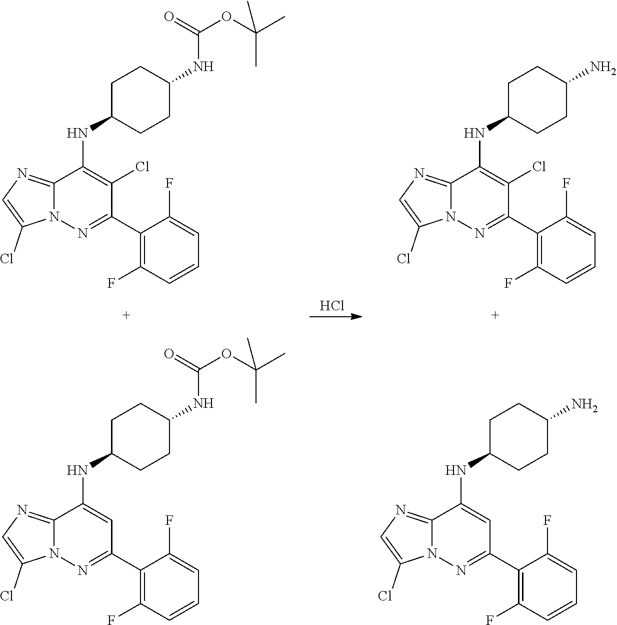 [SMILES: CC(C)(C)OC(=O)N[C@H]1CC[C@H](Nc2c(Cl)c(-c3c(F)cccc3F)nn3c(Cl)cnc23)CC1 ; N[C@H]1CC[C@H](Nc2c(Cl)c(-c3c(F)cccc3F)nn3c(Cl)cnc23)CC1 ; Cl ; CC(C)(C)OC(=O)N[C@H]1CC[C@H](Nc2cc(-c3c(F)cccc3F)nn3c(Cl)cnc23)CC1 ; N[C@H]1CC[C@H](Nc2cc(-c3c(F)cccc3F)nn3c(Cl)cnc23)CC1]

The mixture from the step before was dissolved in MeOH (dry) and treated with an excess HCl (4M in dioxane). The mixture was stirred at r.t., until completion, then concentrated to dryness and purified by reversed phase HPLC.

The compounds of Examples #8, and #9 were synthesized using this procedure.

The amines used were either commercially available, described in the literature, or made by one of the following procedures:

### A: 4-(piperidin-1-yl)cyclohexanamines

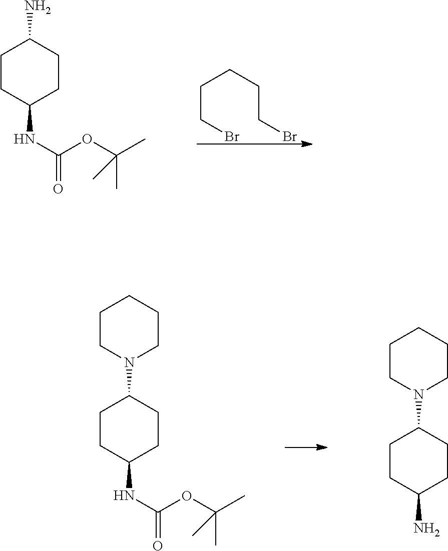 [SMILES: CC(C)(C)OC(=O)N[C@H]1CC[C@H](N)CC1 ; BrCCCCCBr ; CC(C)(C)OC(=O)N[C@H]1CC[C@H](N2CCCCC2)CC1 ; N[C@H]1CC[C@H](N2CCCCC2)CC1]

Step 1: The boc-protected diamine was dissolved in acetonitrile. Dibromo pentane (2 eq.) and DIPEA (5 eq.) were added and the mixture stirred at r.t. o.n.. The mixture was diluted with EtOAc and washed with ammonium chloride (aq. sat.) and water. The organic layer was dried (Na₂SO₄), filtered and concentrated under reduced pressure. The crude product was purified by reversed phased HPLC.

Step 2: The product from step 1 was dissolved in MeOH and treated with HCl (4M in dioxane, excess) o.n.. The mixture was concentrated and used without further purifications as HCl salt.

The following intermediates were synthesized following these protocols.

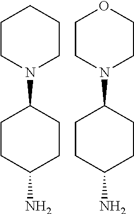

The following examples were prepared according to the procedures described above:

### Example: Name; HPLC Method; r.t.; MH+; NMR

Example #1:3-chloro-6-(2,6-difluorophenyl)-N-((1r,4r)-4-(4-methylpiperazin-1-yl)cyclohexyl)imidazo[1,2-b]pyridazin-8-amine; “HPLC-MS C”; 3.653 min; 461.2;
Example #3:3-chloro-6-(2,6-difluorophenyl)-N-((4,6-dimethylpyridin-3-yl)methyl)imidazo[1,2-b]pyridazin-8-amine; “HPLC-MS C”; 3.93 min; 400.2; “1H NMR (600 MHz, Chloroform-d) δ 8.39 (s, 1H), 7.46 (s, 1H), 7.41 (tt, J=8.4, 6.2 Hz, 1H), 7.07-6.99 (m, 3H), 6.21 (s, 1H), 5.91 (t, J=5.2 Hz, 1H), 4.46 (d, J=5.1 Hz, 22H), 2.53 (s, 3H), 2.35 (s, 3H).”
Example #4:3-chloro-6-(2,6-difluorophenyl)-N-(pyridin-3-ylmethyl)imidazo[1,2-b]pyridazin-8-amine; “HPLC-MS C”; 3.67 min; 372.0; “1H NMR (600 MHz, Chloroform-d) δ 8.64 (d, J=2.4 Hz, 1H), 8.60-8.56 (m, 1H), 7.70 (dt, J=7.9, 2.0 Hz, 1H), 7.50 (s, 1H), 7.39 (tt, J=8.5, 6.3 Hz, 1H), 7.31 (dd, J=7.9, 4.8 Hz, 1H), 7.04-6.97 (m, 2H), 6.34 (d, J=6.0 Hz, 1H), 6.12 (s, 1H), 4.59 (d, J=5.7 Hz, 2H).
Example #8: (1r,4r)-N1-(3,7-dichloro-6-(2,6-difluorophenyl)imidazo[1,2-b]pyridazin-8-yl)cyclohexane-1,4-diamine; “HPLC-MS C”; 3.737 nmin; 412.2; “1H NMR (600 MHz, Chloroform-d) δ 7.54 (s, 1H), 7.44 (tt, J=8.5, 6.3 Hz, 1H), 7.05-6.99 (m, 2H), 5.17 (m, 2H, CH and NH), 4.94 (broad s, 2H, NH2), 2.98 (d, J=12.0 Hz, 1HH), 2.33 (d, J=12.6 Hz, 2H), 2.15 (d, J=12.7 Hz, 2H), 1.72-1.60 (m, 2H), 1.44-1.34 (m, 2H).”
Example #9: (1r,4r)-N1-(3-chloro-6-(2,6-difluorophenyl)imidazo[1,2-b]pyridazin-8-yl)cyclohexane-1,4-diamine; “HPLC-MS C”: 3.358 min; 378.2; “1H NMR (800 MHz, Chloroform-d) δ 7.84 (d, J=1.1 Hz, 1H), 7.62 (d, J=1.2 Hz, 1H), 7.46 (tt, J=8.3, 6.2 Hz, 1H), 7.09-7.02 (m, 2H), 5.23 (t, J=10.9 Hz, 1H), 5.18 (d, J=9.0 Hz, 1H, NH), 3.86-3.61 (broad m, 2H, NH2), 2.93 (s, 1H), 2.34 (d, J=12.4 Hz, 2H), 2.11 (d, J=16.1 Hz, 2H), 1.59 (q, J=12.7, 11.9 Hz, 2H), 1.45-1.38 (m, 2H).”
Example #10:3-chloro-6-(2,6-difluorophenyl)-N-(1-methylpiperidin-4-yl)imidazo[1,2-b]pyridazin-8-amine; “HPLC-MS C”; 3.480 min; 378.2;
Example #11:3-chloro-6-(2,6-difluorophenyl)-N-((1r,4r)-4-morpholinocyclohexyl)imidazo[1,2-b]pyridazin-8-amine; “HPLC-MS C”; 3.763 min; 448.2; “1H NMR (800 MHz, Chloroform-d) δ 7.49 (s, 1H), 7.43 (td, J=8.7, 4.3 Hz, 1H), 7.05 (t, J=7.8 Hz, 2H), 6.10 (s, 1H), 5.72 (d, J=8.0 Hz, 1H), 3.76-3.73 (m, 4H), 3.44 (s, 11H), 2.59 (s, 4H), 2.29 (s, 3H), 2.07-2.04 (m, 2H), 1.43-1.38 (m, 4H).”
Example #12: (1r,4r)-N1-(3-chloro-6-(2,6-difluorophenyl)imidazo[1,2-b]pyridazin-8-yl)-N4,N4-dimethylcyclohexane-1,4-diamine; “HPLC-MS C”; 3.630 min; 406.2;
Example #14:3-chloro-6-(2,6-difluorophenyl)-N-(3-methyl-1-(methylsulfonyl)piperidin-4-yl)imidazo[1,2-b]pyridazin-8-amine“HPLC-MS C”; 4.021 min; 456.2.
Example #15:3-chloro-6-(2,6-difluorophenyl)-N-(3-methylpiperidin-4-yl)imidazo[1,2-b]pyridazin-8-amine; “HPLC-MS C”; 3.562 min; 378.2.
Example #16: tert-butyl-4-((3-chloro-6-(2,6-difluorophenyl)imidazo[1,2-b]pyridazin-8-yl)amino)-3-methylpiperidine-1-carboxylate; “HPLC-MS C”; 4.698 min; 478.2.
Example #17:3-chloro-6-(2,6-difluorophenyl)-N-(1-(methylsulfonyl)piperidin-4-yl)imidazo[1,2-b]pyridazin-8-amine; “HPLC-MS C”; 3.889 min; 442.2.
Example #18:3-chloro-6-(2,6-difluorophenyl)-N-(1-methylpyrrolidin-3-yl)imidazo[1,2-b]pyridazin-8-amine; “HPLC-MS C”; 3.416 min; 364.2.
Example #19:3-chloro-6-(2,6-difluorophenyl)-N-(1-methylazetidin-3-yl)imidazo[1,2-b]pyridazin-8-amine; “HPLC-MS C”; 4.453 min; 350.2; “1H NMR (400 MHz, Chloroform-d) δ 7.52 (s, 1H), 7.41 (tt, J=8.2, 6.2 Hz, 1H), 7.08-6.99 (m, 2H), 6.03 (s, 1H), 4.50 (dd, J=14.5, 7.0 Hz, 1H), 4.10 (dd, J=14.2, 5.6 Hz, 1H), 3.26 (s, 3H), 3.22 (s, 2H), 3.14 (d, J=11.9 Hz, 1H), 2.67 (t, J=11.1 Hz, 1H).”

### Biological Data

### ASSAY PROTOCOL for Determination of Inhibitory Activity

The testing compound was evaluated in 12 concentrations in a 1:3 dilution series, starting from 5microM as the highest concentration.

In a white 384 microplate (Greiner bio-one, Austria #784904,) with 2 microL of 3-fold concentrated CDK9/CyclinT1 (ProQinase/Reaction Biology, USA #0371-0345-1, LOT.: 012), final concentration (f.c.) 6 nM, in 1× kinase buffer, 2 microL of compound (3× fold concentrated in f.c. 1.66% DMSO/H2O) was added and incubated at Room Temperature (RT) for 10 min. Then 2 microL of Substrate/ATP-Mix (3× PDKtide f.c. 40 microM, SignalChem Biotech, Canada via Biozol, Eching, Germany #P10-58, LOT.: L2230-7 and 3× UltraPure ATP f.c. 10 microM (ADP Glo Kinase assay, Promega GmbH, Germany #V9102)) were added, mixed and incubated for 120 min at RT.

After incubation time, 5 microL of ADP Glo reagent (ready to use, ADP Glo Kinase assay, Promega GmbH, Germany #V9102) were added, mixed and incubated for 40 min at RT.

In the last step, 10 microL of ADP Glo Detection reagent (ready to use, ADP Glo Kinase assay, Promega GmbH, Germany #V9102) were added, mixed and incubated for 30 min at RT.

GloMax Discover GM3000 Reader (Promega GmbH, Germany 9700000249) was used for the readout.

The inhibitor concentration was plotted against the luminescence to determine the IC50 using XLFit 5.5 (IDBS, Guildford) to fit to a sigmoidal dose response curve with a variable slope.

### CDK9/T1 ADPGlo Activity of the Compounds of the Above-Described Examples:

IC50<20 nM: Example #3, Example #4, Example #10, Example #11, Example #17, Example #18.
IC50<200 nM: Example #1, Example #8, Example #9, Example #12, Example #14, Example #15, Example #16.

1. A compound of formula (I): 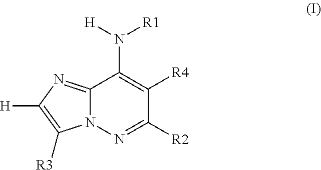
wherein
R1 is an alkyl, alkenyl, alkynyl, heteroalkyl, cycloalkyl, heterocycloalkyl, alkylcycloalkyl, heteroalkylcycloalkyl, aryl, heteroaryl, aralkyl or heteroaralkyl group; all of which groups may optionally be substituted;
R2 is an optionally substituted phenyl group;
R3 is a halogen atom;
R4 is a hydrogen atom or a halogen atom;
or a salt thereof.

2. A compound according to claim 1, wherein R4 is H or Cl.

3. A compound according to claim 1, wherein R4 is H.

4. A compound according to claim 1, wherein R³ is Cl

5. A compound according to claim 1, wherein R2 is a phenyl group that is substituted by 1, 2 or 3 substituents that are independently selected from F, Cl, Br and Me.

6. A compound according to claim 1, wherein R2 is a 2,6-difluorophenyl group.

7. A compound according to claim 1, wherein R1 is an optionally substituted C₃₋₇ cycloalkyl group, an optionally substituted heterocycloalkyl group containing from 3 to 9 ring atoms that are independently selected from C, N and O, an optionally substituted phenyl group, an optionally substituted benzyl group, an optionally substituted heteroaryl group containing 5 or 6 ring atoms that are independently selected from C, N, O and S, or a group of formula —CH₂-Het, wherein Het is an optionally substituted heteroaryl group containing 5 or 6 ring atoms that are independently selected from C, N, O and S.

8. A compound according to claim 1, wherein R1 is an optionally substituted C₃₋₇ cycloalkyl group, an optionally substituted heterocycloalkyl group containing from 3 to 9 ring atoms that are independently selected from C, N and O, or a group of formula —CH₂-Het, wherein Het is an optionally substituted heteroaryl group containing 5 or 6 ring atoms that are independently selected from C, N, O and S.

9. A compound according to claim 1, wherein R1 is selected from the following groups: 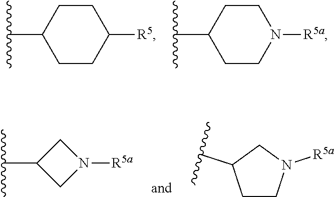
wherein R⁵ is NH₂, a C₁₋₈ heteroalkyl group, or an optionally substituted heterocycloalkyl group containing 5 or 6 ring atoms that are independently selected from C, N and O; and R^{5a} is hydrogen, a C₁₋₆ alkyl group, a C₁₋₈ heteroalkyl group, an optionally substituted C₅₋₆ cycloalkyl group or an optionally substituted heterocycloalkyl group containing 5 or 6 ring atoms that are independently selected from C, N and O.

10. A compound according to claim 1, wherein R1 is selected from the following groups: 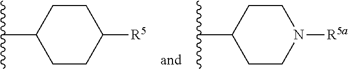
wherein R⁵ is NH₂, a C₁₋₆ heteroalkyl group, or an optionally substituted heterocycloalkyl group containing 5 or 6 ring atoms that are independently selected from C, N and O; and R^{5a} is hydrogen, a C₁₋₆ alkyl group, an optionally substituted C₅₋₆ cycloalkyl group or an optionally substituted heterocycloalkyl group containing 5 or 6 ring atoms that are independently selected from C, N and O.

11. A compound according to claim 1, wherein R1 is a group of formula —CH₂-Het wherein Het is an optionally substituted pyridyl group.

12. A compound according to claim 1 which is selected from the following compounds or a salt thereof:
3-chloro-6-(2,6-difluorophenyl)-N-((1r,4r)-4-(4-methylpiperazin-1-yl)cyclohexyl)imidazo[1,2-b]pyridazin-8-amine;
3-chloro-6-(2,6-difluorophenyl)-N-((4,6-dimethylpyridin-3-yl)methyl)imidazo[1,2-b]pyridazin-8-amine;
3-chloro-6-(2,6-difluorophenyl)-N-(pyridin-3-ylmethyl)imidazo[1,2-b]pyridazin-8-amine;
(1r,4r)-N1-(3,7-dichloro-6-(2,6-difluorophenyl)imidazo[1,2-b]pyridazin-8-yl)cyclohexane-1,4-diamine;
6 Docket No.: 348710.02101 (1r,4r)-N1-(3-chloro-6-(2,6-difluorophenyl)imidazo[1,2-b]pyridazin-8-yl)cyclohexane-1,4-diamine;
3-chloro-6-(2,6-difluorophenyl)-N-(1-methylpiperidin-4-yl)imidazo[1,2-b]pyridazin-8-amine;
3-chloro-6-(2,6-difluorophenyl)-N-((1r,4r)-4-morpholinocyclohexyl)imidazo[1,2-b]pyridazin-8-amine;
(1r,4r)-N1-(3-chloro-6-(2,6-difluorophenyl)imidazo[1,2-b]pyridazin-8-yl)-N4,N4-dimethylcyclohexane-1,4-diamine;
3-chloro-6-(2,6-difluorophenyl)-N-(3-methyl-1-(methylsulfonyl)piperidin-4-yl)imidazo[1,2-b]pyridazin-8-amine;
3-chloro-6-(2,6-difluorophenyl)-N-(3-methylpiperidin-4-yl)imidazo[1,2-b]pyridazin-8-amine;
tert-butyl-4-((3-chloro-6-(2,6-difluorophenyl)imidazo[1,2-b]pyridazin-8-yl)amino)-3-methylpiperidine-1-carboxylate;
3-chloro-6-(2,6-difluorophenyl)-N-(1-(methylsulfonyl)piperidin-4-yl)imidazo[1,2-b]pyridazin-8-amine;
3-chloro-6-(2,6-difluorophenyl)-N-(1-methylpyrrolidin-3-yl)imidazo[1,2-b]pyridazin-8-amine; and
3-chloro-6-(2,6-difluorophenyl)-N-(1-methylazetidin-3-yl)imidazo[1,2-b]pyridazin-8-amine.

13. A pharmaceutical composition comprising a compound according to claim 1 and optionally one or more carrier substances and/or one or more adjuvants.

14. (canceled)

15. A method for treating a subject suffering from or susceptible to a disease in which abnormal CDK, especially CDK9 and/or CDK2 regulation is observed, the method comprising:
administering to the subject an effective amount of a compound of claim 1.

16. The method of claim 15 wherein the subject is identified as suffering from or susceptible to the disease and the compound is administered to the identified subject.

17. The method of claim 15 wherein the subject is suffering form or susceptible to cytokine-induced inflammatory, and autoimmune diseases, local or systemic viral infection diseases, viral infections of the eye, viral respiratory infections, or viral infections of the central and/or peripheral nervous system caused by DNA and/or RNA viruses, and various non-solid and solid malignancies, cancers, or hyperproliferative diseases such as acute myelogenous leukemia, chronic lymphocytic leukemia, relapsed multiple myeloma, non-Hodgkin's lymphoma, acute lymphoblastic leukemia, acute biphenotypic leukemia, aggressive MYC-driven B-Cell lymphoma, primary peritoneal carcinoma, Kaposi sarcoma, advanced breast cancer, non-small cell lung cancer, colorectal cancer, or liver cancer such as hepatocellular carcinoma, cervical intraepithelial neoplasia, prostate cancer, melanoma, glioma, glioblastoma, neuroblastoma, astrocytoma, anaplastic astrocytoma or glioblastoma including advanced and/or metastatic haematological/solid malignancies; mechanical/injury-induced inflammation such as post-traumatic osteoarthritis (PTOA), systemic and local cytokine-induced inflammatory disease, including gastrointestinal or urinary tracts inflammatory diseases inflammatory diseases and inflammatory diseases of the eye, such as Sjogren's disease and glaucoma, bacteria-induced inflammatory disease, such as gingivitis, periodontitis, and/or a cardiovascular disease such as cardiac hypertrophy, dilated cardiomyopathy, atherosclerosis, and/or a cardio-metabolic disease such as obesity and diabetes.

18. The method of claim 17 wherein the subject is identified as suffering from or susceptible to the disease and the compound is administered to the identified subject.
